(19) United States
(12) Patent Application Publication
(10) Pub. No.: US 2002/0076402 A1
(43) Pub. Date: Jun. 20, 2002

(54) Enzyme-based fungicidal composition

(75) Inventors: Chadi Khouri, Zahle (LB); Michel Minier, Antony (FR); Francois Le Goffic, Clamart (FR); Nguyen Van Huynh, Liege (BE)

Correspondence Address: FITCH EVEN TABIN AND FLANNERY, 120 SOUTH LA SALLE STREET, SUITE 1600, CHICAGO, IL, 60603-3406, US

(73) Assignee: Universite Pierre et Marie Curie

(21) Appl. No.: 09/843,169

(22) Filed: Apr. 26, 2001

Related U.S. Application Data
(63) Continuation of application No. PCT/FR99/02645, filed on Oct. 28, 1999.

(30) Foreign Application Priority Data
Oct. 28, 1998 (FR) 9813530

(51) Int. Cl. A61K 38/47; A01N 25/00

(52) U.S. Cl. 424/94.61; 424/405

(57) ABSTRACT

The invention concerns a fungicidal or fungistatic composition comprising, in combination, at least one glycolytic enzyme and its substrate and/or oligomers thereof; a process for preparing said compositions; and their use.

The present invention relates to fungicidal compositions using mixtures of enzymes and natural substrates acting in synergy to inhibit fungal growth, and which can be applied to different types of surfaces in the form of a solid or semi-solid coating.

It also relates to a process for preparing such compositions and to their use, in particular for protecting seeds against phytopathogenic fungi.

The bibliographic references cited in brackets in the present text are listed at the end of the description.

The most routinely used antifungal substances, whether for medical use or for agricultural use, are molecules that are chemically synthesised, which often have non negligible toxicity towards mammals, fowl, fish and other living organisms (Index phytosanitaire 1995, Ed. ACTA).

For agricultural use, they have the disadvantage of their being capable of entering the water table and of being found in food. Their broad spectrum of action has the disadvantage of eliminating species known to be useful in that they participate in limiting the emergence of other causes of havoc. The adaptive ability of some fungal species of insects to be eradicated should also be mentioned.

In contrast, evolution has endowed higher plants with highly complex defence systems involving cascades of events controlled by mediators and effectors that may be proteins, polyosidic, or low molecular weight molecules. Synergistic effects exist in these natural defence reactions between several families of enzymes and different small antifungal molecules: phytoalexins, antibiotics (Schirmböck et al., 1994; Rajnchapel-Messaï, 1988). As an example, certain plants react to insect or fungal attack by producing enzymes, chitinases or glucanases, which are capable of degrading the polysaccharide walls of those attackers. The composition of fungal walls, constituted by interleaved organised layers of a variety of polysaccharides, such as poly-β, 1-3 glucanes and chitin (i.e., poly-β, 1-4-N-acetylglucosamine) renders such a complex response necessary.

One disadvantage is that the defence capacity is not shared by all plant species or varieties and that it has to be triggered by first attacking the plant, hence the need to provide the plant with supplemental protection.

A first approach that is currently being studied uses genetic engineering and consists of cloning into the plant the gene for a protein involved in such defence reactions, for example a chitinase, a glucanase or a further enzyme that can inhibit the growth of phytopathogenic fungi (Gilbert et al., 1996; Cornelissen and Melchers, 1993; Stintzi et al., 1993; Harman et al., 1992). The following can be cited: the introduction of the gene for a bean chitinase into tobacco and into transgenic rapeseed, which endowed the plants with increased resistance to Rhizoctonia solani (Broglie et al., 1991). Similarly, the introduction of a gene for the bacterium Serratia marcescens into transgenic tobacco produced similar results (Jach et al., 1992). In contrast, the over-expression of a tobacco chitinase in a transgenic tobacco was achieved with no demonstration of any significant protective effect (Neuhaus et al., 1991).

The role of lysozyme was also mentioned insofar as it has an endochitinase activity and where many plant enzymes have a lysozyme/chitinase bifunctionality (Düring, 1993). Hen lysozyme was cloned into transgenic tobacco then produced and secreted by the plant and its activity was measured in vitro: under those conditions, the recombined enzyme was capable of inhibiting certain bacteria or fungi (Trudel et al., 1995); however, protection of the plant was not demonstrated.

The results obtained are still variable depending on the coupling constituted by the plant variety and the microbial strain.

In summary, the main disadvantages of the plant genetic engineering method are:

the relatively long time period required to develop a recombined plant variety;

the increased difficulty of introducing and rendering effective a number of enzyme genes, for example;

the difficulty of generalising the knowledge gained in recombinant work carried out on a plant to other species or varieties;

the risks, proven or otherwise, attached to using recombinant plants outside the laboratory.

A second approach consists of attempting to introduce the physiological reactions of the plant itself: defence against potential attackers or against germination.

Certain plants are known to react to the presence of chitin, chitosan or oligosaccharides produced by degradation of the wall of their own cells or the degradation of fungal walls (Teichgräber et al., 1991).

It is also possible to cite the increased production of chitinases by different seeds that are soaked in solutions containing chitosan and chitosan derivatives (Hirano et al., 1990, Teichgräber et al., 1991).

Similarly, in cultures of rice cells in suspension in a liquid medium, Inui et al. (1996) demonstrated that the presence of N-acetyloligosaccharides increased the production of chitinases by such cells.

The examples cited above do not, however, provide answers to the question of protecting a seed during the period between its harvest and sowing, taking into account the usual agricultural practices (immersing seeds before storage is not practical).

Preparations containing compounds such as chitin, chitosan and derivatives thereof, N-acetylglucosamine, have demonstrated their ability to encourage seed germination (He et al., 1990). However, in that case, there is no protection of the seeds during storage and during the phases preceding awakening of the metabolic activity and germination. Competition between the speed of any attack and the defensive response of the seed alone controls the efficiency of that method as regards pathogens.

The use of a chitin-protein mixture known as Clandosan 618 and sold by IGENE Biotechnology Inc. (Columbia Md., USA) as a nematicide can also be cited, but that requires its incorporation into the ground in quantities of the order of two tonnes per hectare U.S. Pat. No. 5,057,141 ).

Finally, a third approach proposed for the protection of seeds is their protection using bacteria.

It consists of producing a casing or pellicule or by encapsulation that can immobilise certain beneficial bacteria on the seed during the storage period. Once sown, those bacteria can protect the plant by colonising the rhizosphere at the expense of pathogenic telluric germs by simple occupation of the biotope, possibly assisted by secretion of antibiotic substances; in some cases they can also produce substances that stimulate germination or growth of the plant by effects comparable to that of certain hormones. The most frequently studied bacteria for these purposes that can be cited are Pseudomonas fluorescens and P. putida (Digat, 1994).

However, that technique suffers from certain disadvantages:

the bacteria brought into contact with the seeds undergo the same treatments as the seeds themselves: drying, and in some cases heating. After this hydration and heat “stress” and a storage period of several months, the bacteria are not always capable of developing when faced with competition from telluric flora;

when rhizosphere colonisation is effective, there is the possibility of an abusive introduction of living species into the environment, and the risk attached thereto is unknown.

The following can also be cited: preparations containing chitin, a culture of bacteria of the genus Streptomyces and perlite which, mixed with vegetable mould and compost, protects against the garlic mosaic virus (Kajimura et al., 1991). However, that example is far from the envisaged application and also suffers from the disadvantage of having to handle live bacteria.

The present invention describes compositions employing active substances that are natural, non polluting, non toxic to man or to animals, directly applicable in the context of usual agricultural practices, or in other applications, and provide active protection against phytopathogenic fungi or other fungi, while maintaining or improving seed germination ability.

These compositions are applied directly to the articles to be protected, in particular seeds, bulbs or roots.

It consists of a carefully selected combination of at least one glycolytic enzyme and its substrate and/or oligomers thereof in proportions which, when the composition is applied to seeds, have a germination inhibiting effect of less than 10%.

The glycolytic enzyme or enzymes are obtained by production from non pathogenic bacterial strains that are non recombinant and can be cultivated in a fermenter. The enzymes in question taken in isolation or in pairs, only provide a very small amount of protection against contamination by phytopathogenic fungi; further, they reduce the germination capacity of seeds, as will be demonstrated in the examples below.

In the compositions of the invention, the glycolytic enzyme or enzymes are combined with other enzymes or polysaccharide compounds incubated with enzymes which alone do not exhibit any significant antifungal activity but which accelerate seed germination, as will be shown in Examples 3 and 4 below.

Preferably, the glycolytic enzyme or enzymes are glycosidases selected from the group formed by chitinases and laminarinases.

The other enzymes or polysaccharide compounds that can advantageously be incorporated into the composition of the invention are lysozyme and a chitin or chitosan or oligomers thereof obtained by controlled hydrolysis.

Finally, the compositions of the invention can comprise a film-forming agent to enable them to be applied to the article to be protected.

The term “carefully selected” as used in the context of the combination of the invention means that selection of each of the enzymatic elements of the polysaccharide substrate and, if necessary, the film-forming compound is such that the seeds to be protected can be directly treated using a method that will be described below, so as to have both a degree of fungal growth inhibition of more than 60%, preferably 80% and a zero positive germination promotion effect or an inhibiting effect of a maximum of 10%.

The properties characterising certain combinations do not occur as an addition of the properties of the different elements but as a synergistic effect involving inhibition reactions directly applied to phytopathogenic fungi, and the transmission of mediators involved in the defence and germination processes of the seed.

The preparations comprised, for example, of chitinases of the bacterium Serratia marcescens, laminarinases from the bacterium Bacillus circulans, hen lysozyme and chitin partially hydrolysed by said chitinases, have caused a very substantial reduction in the contamination of the seeds, much greater than the effects obtained with incomplete formulae, and retain their germination capacity.

This result was obtained with seeds contaminated by phytopathogenic fungi, coated with commercially available preparations intended for pelliculisation into which certain of the combinations cited above have been incorporated, then dried under the conditions prevailing in agriculture (40° C. air stream) and deposited in Petri dishes containing gel media that assist fungal development and seed germination. It can then be shown that, under realistic conditions of use, the combinations of enzymes and natural substrates necessary to inhibition of phytopathogenic fungi and to the promotion of germination remains active and mobilisable after bringing the pelliculisation agents into contact and drying the grain. Further, tests for storage at different temperatures, after drying, of the pelliculisation agent containing chitinases have demonstrated that the stability and activity of the chitinases are retained or even improved even after a period of several months.

A preferred combination of the invention preferably comprises a chitinase, a laminarinase, lysozyme and a chitin and/or oligomers thereof obtained by controlled hydrolysis.

Preferred oligomers originating from chitin have the formula [N-acetylglucosamine]ₙ, or (NAG)ₙ, n being in the range 1 to 8, and more preferably n=2 or 3.

Other polysaccharides resulting from chitin deacetylation must be considered as functional equivalents thereof, also their controlled hydrolysis products. An example is chitosan, a 100% deacetylated derivative of chitin.

In the composition of the invention, the ratio by weight of the enzymes to the glycosaccharides can be in the range 10/1 to 1/10.

The desired effect as a fungicide and germination stimulant can be achieved when 100 mg of chitinases is combined with 10 to 1000 mg of chitin or partial hydrolysis products thereof.

The compositions of the invention can also comprise film-forming preparations, in particular pelliculisation, enrobing or encapsulation agents.

In the description, the term “pelliculisation” or “enrobing” is used to describe a coating that adheres to the grain. The coating can, for example, be deposited on the grain in the form of an aqueous mush and the whole can be dried in hot air (40° C.-45° C.). Any industrial process used for pelliculisation or enrobing can be used for pelliculisation and enrobing of the compositions of the invention.

The pelliculisation agents are commercially available products: Sepiret 01 G and Sepiret 7017 Argent (Seppic, Castres, France). Sepiret 01 G is sold in the form of a powder while Sepiret 7017 Argent is a thick suspension containing 29% dry matter.

These film-forming preparations must be suitable for their application to different types of surfaces in the form of a solid or semi-solid coating. Non-limiting examples of articles to be treated by these compositions that can be cited are seeds, bulbs and roots that may be stored. In the same manner, seeds from previously treated seeds are durably protected against infections.

The compositions can also be formed and used by spraying onto small areas such as apartment plants or biological gardens.

In the food industry, the compositions can be employed to preserve food by using active packaging coatings.

Other pelliculisation or enrobing agents can be used. The criteria which govern the choice of these agents are: the texture conferred on the composition to be applied, the absence of effects on the enzymatic activities of the composition and the absence of an effect on the physiology of the agricultural products when applied to them.

The present invention also concerns a method for preparing a fungicidal or fungistatic composition comprising the following steps:

a) producing glycosidases by microbial or yeast culture;

b) extracting the enzymes;

c) mixing the enzymatic preparations with their natural substrate or derivatives thereof obtained by controlled hydrolysis;

d) incorporation, if necessary, into a film-forming pelliculisation or enrobing type agent;

e) if necessary, verifying the activity by incubation in the presence of pathogenic fungi.

In the method of the invention, the chitinases can, for example, be produced by culturing Serratia marcescens, and laminarinase can be produced by culturing Bacillus circulans.

More particularly, chitinase can be produced from the Serratia marcescens strain deposited on Oct. 15^{th}, 1998 at the BCCM [BCCM/LMG: Collection of the Laboratorium voor Microbiologie en Microbiele Genetica, Ghent, Belgium] with accession number LMGP-18541, and is included in the scope of the invention.

The preparation can also contain a lysozyme, which may be a commercially available lysozyme.

As will be shown in Example 3 below, lysozyme can improve the degree of germination.

The natural substrates are chitin (poly β 1-4-N-acetylglucosamine) and chitin oligomers obtained by controlled hydrolysis of the chitinases cited above.

Preferred oligomers originating from chitin have the formula [N-acetylglucosamine]ₙ, or (NAG)ₙ, n being in the range 1 to 8 and more preferably n=2 or 3.

The choice of enzymes and their relative proportions will be guided by fungus culture tests under the following conditions:

The gel growth medium is composed of, in g/l: 2 yeast extract; 1.5 KH₂PO₄; 1.5 K₂HPO₄; 0.3 Mg(SO₄).7H₂O; 0.3 NaNO₃; and 14 agar. Culture is carried out at 16° C. in Petri dishes closed with cling film.

The prepared compositions are then deposited into Petri dishes.

The fungicidal effect is then measured by inhibiting the growth of fungal hyphae, and compared with preparations completely or partially depleted in certain of the constituents.

When the test is applied to pelliculised grains or seeds, the degree of germination is determined by the number of grains germinated over the total number of grains, and compared with those of non pelliculised batches, or grains that are pelliculised grains in the absence of enzymes and/or their substrate.

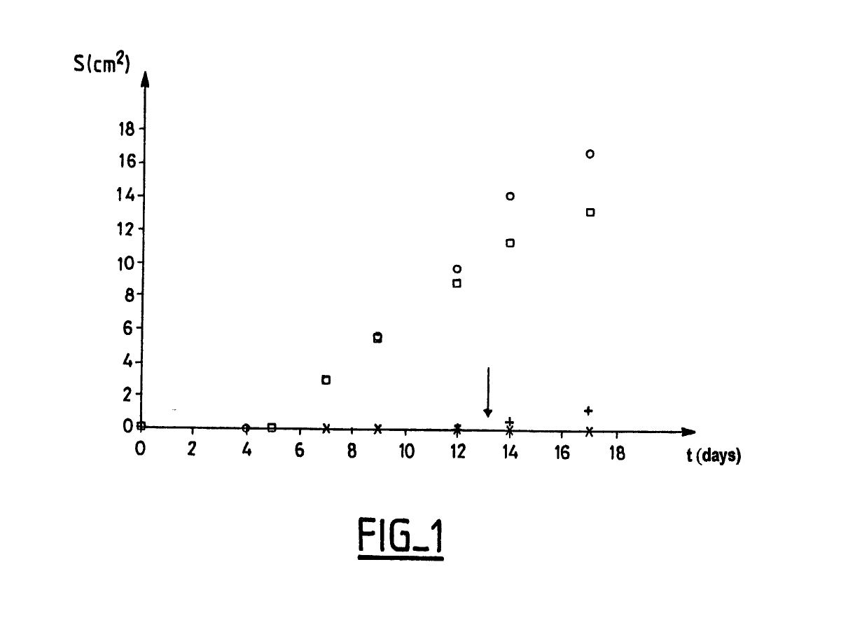

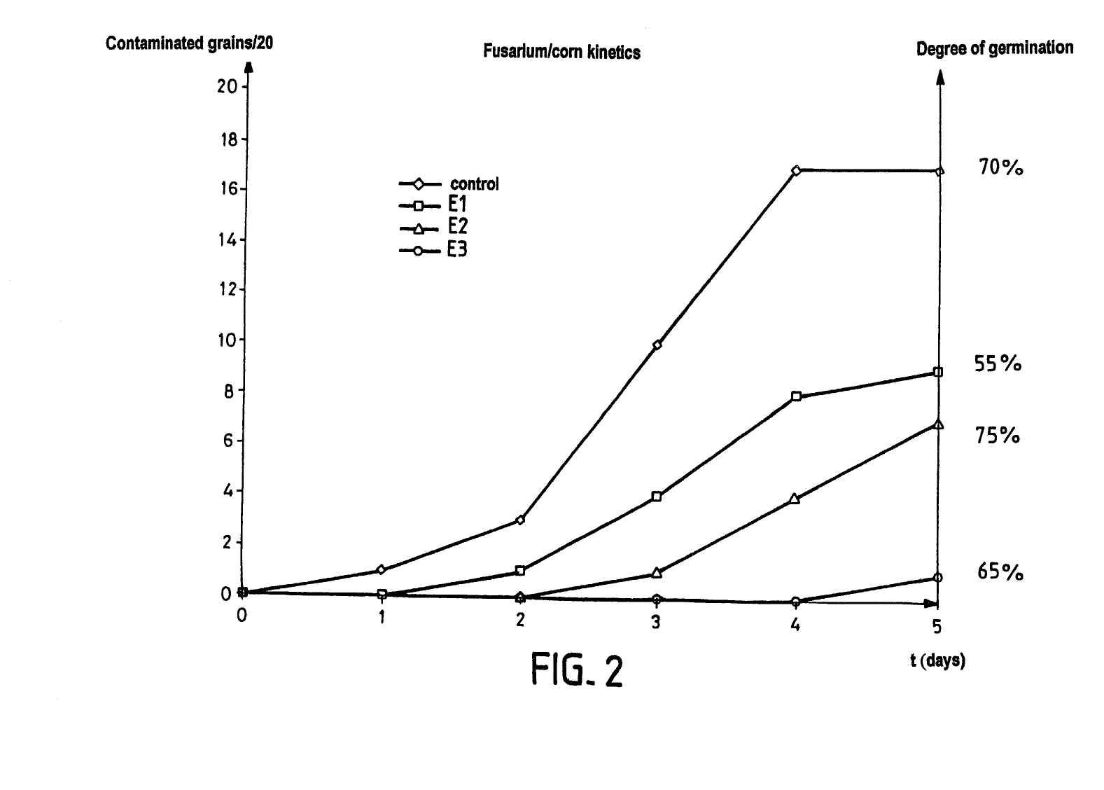

Examples will now be given of the preparation of the constituents of the composition of the invention, of the fungicidal effect and of the effect on seed germination of the preparations, made with reference to FIGS. 1 and 2.

FIG. 1 shows a standardised Botrytis inhibition test using chitinases. (0) and ( ) are control cultures and (X) and (+) are treated fungi. The arrow indicates the onset of growth of one of the treated fungi.

FIG. 2 shows the contamination kinetics and the degree of germination as a function of the applied mixture. It illustrates the results of Example 5 below.

The skilled person will be able to adapt the methods of the examples to other enzyme/substrate combinations.

Method and Apparatus

1. Chitinase Production and Recovery

The chitinases described below were produced in a fermenter from a Serratia marcescens strain deposited at the BCCM with accession number LMGP-18541 on Oct. 15^{th}, 1998, cultivated on a medium containing (in g/l): 10 chitin; 0.5 yeast extract; 1 (NH₄)₂SO₄; 0.3 Mg(SO₄).7H₂O and 1.36 KH₂PO₄. Following fermentation (4 to 5 days), the must was freed of bacteria by centrifuging then filtering at 0.2 μm and the proteins were precipitated with ammonium sulphate (70% saturated). The residue was recovered by centrifuging, dialysed against a 200 mM phosphate buffer at a pH of 6.6, then freeze-dried. The powder obtained was termed Chitinases or CNase.

2. Laminarinase Production and Recovery

The laminarinases described in the invention were produced in a fermenter using ATCC 21367 Bacillus circulans strain in a medium containing (in g/l): 12 chitin; 1 yeast extract; 0.2 glucose; 2 KH₂PO₄; 0.2 Mg(SO₄).7H₂O; and 1 (NH₄)₂SO₄. After freeing from bacteria, the must was concentrated by frontal ultrafiltration (Amicon module and YM10 filter) then dialysed against an acetate buffer at a pH of 5. The solution obtained was termed Laminarinases (LMase).

3. Lysozyme

The lysozyme used, (LZ) in this description was a commercially available lysozyme hydrochloride powder (Sigma).

4. Chitin

Treatment of unrefined chitin (Sigma) with hydrochloric acid then hot sodium hydroxide then phosphoric acid produced colloidal chitin purified of calcium ions and the proteins initially present.

5. Preparation of Chitinase/Chitin Mixture

100 mg of CNase and 200 mg of colloidal chitin in 10 ml of phosphate buffer at a pH of 6.6 were incubated at 50° C. for 15 to 20 minutes. The mixture was then freeze-dried and denoted (M+).

It should be understood that mixture (M+) contained active chitinases.

Enzyme denaturing by heating just after incubation followed by freeze-drying produced the mixture (M−).

6. Enzymatic Assays

Chitinases:

Chitinolytic activity tests were carried out at 50° C. with stirring on 0.5 ml of enzymatic solution and 1.5 ml of phosphate buffer containing 20 mg of chitin. After one hour, the reaction was stopped by adding trichloroacetic acid and the liberated reducing ends were revealed by heating with a solution of sodium dinitrosalicylate and analysed by spectrophotometry at 530 nm using an N-acetylglucosamine (NAG) calibration curve.

The chitinolytic activity unit is defined as the quantity of enzymes required to liberate one micromole of NAG equivalents after one hour's incubation at 50° C. and at a pH of 6.6.

Laminarinases:

The laminarinase activity was determined by measuring the quantity of glucose equivalents produced by the enzymatic reaction. 0.2 ml of enzymatic sample was buffered with 0.5 ml of 100 mM acetate buffer at a pH of 5 then incubated at 50° C. for 1 h with 0.3 ml of a 30 g/l solution of laminarine (Sigma). The reaction was stopped using trichloroacetic acid. The protocol for assaying the reducing ends was identical to that used to measure the chitinolytic activity.

The laminarinase activity unit corresponds to liberating one micromole of glucose equivalents in one hour at a pH of 5 and at 50° C.

Total protein assay was determined by the BCA method using calf albumin as the reference.

7. Assay and Characterization of Protein Mixtures Used

The CNase protein mixture was in the form of a freeze-dried powder and contained 5.6 U/mg of chitonolytic activity and 0.28 mg of albumin equivalents/mg.

The LMase protein mixture was a liquid. It contained 87 U/ml of laminarinase activity and 0.6 mg of albumin equivalents/ml.

8. Pelliculisation/enrobing

The term “pelliculisation” or “enrobing” is used in this description to define a coating that adheres to the grain. This coating was deposited on the grain in the form of an aqueous stock and the ensemble was dried in hot air (40° C.-45° C.).

The pelliculisation agents used are commercially available products: Sepiret 01 G and Sepiret 7017 Argent (Seppic, Castres, France). Sepiret 01 G is sold in the form of a powder while Sepiret 7017 Argent is a thick suspension containing 29% dry matter.

In the following examples:

FIG. 1 shows a standardised Botrytis inhibition test using chitinases. (0) and ( ) are control cultures and (X) and (+) are treated fungi. The arrow indicates the onset of growth of one of the treated fungi.

FIG. 2 represents the contamination kinetics and the degree of germination of corn seed as a function of the compositions used. E_{1,} E₂ and E₃ have the meanings given in Example 5 below.

### EXAMPLE NO 1

Pelliculisation of Agar Squares: Inhibition of Botrytis cinerea

Thin (1±0.5 mm) agar squares (5±1 mm sides) were removed from a Petri dish containing a culture of the phytopathogenic fungus Botrytis cinerea. They were coated with a suspension of 15% (m/v) Sepiret 01Gcg 20 mg/g of chitinases; they were then dried in hot air (40-45° C.) for 15-20 minutes.

The pelliculised squares were placed in the centre of a Petri dish containing a growth medium. At intervals, the surface area occupied by growing fugal hyphae was determined semi-quantitatively by tracing onto graph paper.

Comparison of the growth of B. cinerea enrobed with chitinases with a control enrobed with the same suspension without chitinases (duplicate test) produced the profiles of FIG. 1.

It could be seen that:

In the absence of chitinases, and after a latent period of 4 to 5 days, the hyphae occupied a surface area of 17 cm² after 17 days.

In the presence of chitinases, a retardation in growth of at least 10 days was observed as well as a reduction in the growth rate: the slopes of the curves representing the growth kinetics in the presence of chitinases were lower. In the case of the test represented by (x) in FIG. 1, a fungicidal effect was observed—compared with the fungistatic effect of the test shown as (+)—since no fungus growth was noted even after 17 days incubation.

### EXAMPLE NO 2

Pelliculisation of Lentils: Inhibition of Botrytis cinerea:

Formulation: 50 mg of CNase was mixed with 1 g of an aqueous 15% (m/v) Sepiret 01G suspension.

Pelliculisation: batches of 12 grains (416 mg) of lentils naturally contaminated with the phytopathogenic fungus Botrytis cinerea were pelliculised with the above preparation using the method described in paragraph 8 above.

Germination inhibition test: the pelliculised grains were placed in Petri dishes containing a growth medium. On the fifth day, the amount of contamination (number of contaminated grains/total number of grains) and degree of germination (number of germinated grains/total number of grains) were determined and compared with those of the non pelliculised batches (naked grains) and batches coated with Sepiret 01G alone. The results obtained are shown in Table I below:

TABLE I

| | Degree of contamination | Degree of germination |
|---|---|---|
| Naked grains | 50% | 80% |
| Sepiret 01G | 31% | 70% |
| Sepiret 01G + CNase | 9% | 53% |

An inhibiting effect on the growth of B. cinerea (degree of contamination changing from 31% to 9%, i.e., a reduction by a factor of three) due to the action of the chitinases. Further, a drop in the degree of germination was observed (70% to 53%).

### EXAMPLE NO 3

Pelliculisation of Corn Contaminated with Fusarium sp.: Mono-enzymatic Mixtures

Formulation:

Chitinases alone: quantity used to pelliculise 3 to 4 g of corn naturally contaminated with the phytopathogenic fungi Fusarium nivale and Fusarium roseum: 100 mg of CNase was mixed with 3.4 ml of a suspension of Sepiret 7017 Argent composed of 3.6 g of the commercial suspension and 10 ml of water.

Laminarinases alone: quantity used to pelliculise 2 g of contaminated corn: 40 mg of freeze-dried LMase with a filter of 17 units/mg were mixed with 2 ml of a suspension of Sepiret 7017 Argent composed of 3 g of the commercial suspension and 5 ml of water.

Lysozyme alone: quantity used to pelliculise 2 g of contaminated corn: 100 mg of LZ was mixed with 2 ml of a suspension of Sepiret 7017 Argent composed of 5 g of the commercial suspension and 7.1 ml of water.

Germination inhibition test: The pelliculised grains were placed in Petri dishes containing the growth medium. On the fifth day, the degree of contamination (number of contaminated grains/total number of grains) and degree of germination (number of germinated grains/total number of grains) were determined and compared with those of batches pelliculised with Sepiret 01G alone. The results obtained are shown in Table II below:

TABLE II

| | Degree of contamination | Degree of germination |
|---|---|---|
| Control, CNase | 86% | 76% |
| Test, CNase alone | 65% | 50% |
| Control, LMase | 85% | 80% |
| Test, LMase alone | 45% | 60% |
| Control, LZ | 65% | 70% |
| Test, LZ alone | 65% | 85% |

Regarding inhibition of Fusarium sp. Fungi:

It can be seen that lysozyme alone had no effect, that chitinases alone had only a very small effect, within the limits of experimental error, and that laminarinases divided the degree of contamination by a factor of 1.9. This can be explained by the fact that the outermost layers of fungal walls are often constituted by glucanes, degraded by laminarinase, while chitin is hidden in the internal layers.

Regarding the degree of germination:

It can be seen that chitinase and laminarinases tend to reduce germination in that order, dividing the degree of germination by a factor of 1.5 and 1.3 respectively; lysozyme does not change and can even improve (relative variation of +20%) the degree of germination.

### EXAMPLE NO 4

Pelliculisation of Corn: Inhibition of Fusarium sp.: Influence of Different Natural Substrates in the Presence or Otherwise of Chitinases

Formulation: An aqueous suspension of Sepiret 7017 Argent composed of 7 g of commercial slip and 10 ml of water was used. 100 mg of the substances shown below was mixed with 2 ml of the above suspension and this mixture was used to pelliculise batches of 2 g of corn. This latter originated from a culture naturally contaminated with Fusarium nivale and Fusarium roseum:

Products tested: N-acetylglucosamine (NAG), colloidal chitin, (M+) and (M−).

Results: The degree of contamination and germination on day five (see method of Examples 2 and 3) are shown in Table III below:

TABLE III

| | Reference | NAG | Chitin | (M+) | (M−) |
|---|---|---|---|---|---|
| Contamination | 65% | 70% | 45% | 30% | 50% |
| Germination | 80% | 65% | 55% | 55% | 70% |

It can be seen that in the absence of active enzymes in the formula, i.e., in the case where the chitin or its monomer NAG or the mixture (M−) are added, the effects on the contaminating fungal growth are small or non existent.

More precisely, the monomer NAG has no effect on the degree of contamination, while the presence of chitin, or chitin partially degraded by prior incubation with chitinases (mixture (M−)) appeared to cause a slight reduction in contamination, which could have been due to a physiological reaction of the seed itself.

In contrast, a reduction by half in the degree of contamination was observed with the mixture (M+) containing chitinases, chitin and products of their hydrolytic reaction.

However, with this mixture (M+) and also in the presence of chitin alone, a reduction in the degree of germination was observed which was divided by a factor of 1.45.

### EXAMPLE NO 5

Pelliculisation of Corn: Multienzymatic Mixture

Formulation: a pelliculisation mixture (S) was prepared by mixing the commercially available Sepiret 7017 Argent slip with the LMase solution defined in paragraph 2 of the “method and apparatus” section in the proportions: 70 g of slip for 100 ml of LMase.

Mixtures tested: (quantities used to pelliculise 2 g of corn:

E1: 100 mg CNase+2 ml (S);

E2: 100 mg CNase+100 mg LZ+2 ml (S);

E3: 100 mg (M+)+100 mg LZ+2 ml (S);

Control: 2 ml of an aqueous 70% (m/v) suspension of Sepiret 7017.

Results:

The degrees of contamination and germination on day 5 are shown in Table IV:

TABLE IV

| | Control | E1 | E2 | E3 |
|---|---|---|---|---|
| Contamination | 85% | 45% | 35% | 5% |
| Germination | 70% | 55% | 75% | 65% |

The contamination kinetics and the degrees of contamination are shown in FIG. 2.

It can be seen that each of formulations E1, E2 and E3 had a significant inhibiting effect on fungal growth, with an improvement in effectiveness from E1 to E3. The antifungal effect of chitinases and laminarinases alone (formulation E1) remained limited and also reduced the degree of germination.

In contrast, a considerable reduction in the degree of contamination by Fusarium sp was observed (changed from 85% to 5%, i.e., a reduction by a factor of 17) for mixture E3 which contained laminarinases, lysozyme and chitinases as well as partially degraded chitin. The degree of germination in this case remained identical to that of the control batch.

### EXAMPLE NO 6

Stability of Chitinases in Sepiret O1G Pelliculisation Agent

Identical batches composed of 10 mg of CNase and 200 mg of an aqueous suspension of Sepiret 01G (15% m/v) were prepared. The batches were vacuum dried at 40° C. and stored at three different temperatures (4° C.; 16° C.; 32° C.). At intervals, samples were removed and the enzymes were taken up into solution in a phosphate buffer with a pH of 6.6. The chitinolytic activity was determined for each sample and compared with that of a batch of CNase (without Sepiret) that had undergone the same treatments (drying then storing at the same temperatures).

The results were as follows: (Table V):

TABLE V

| | | Activity at | Activity at |
|---|---|---|---|
| | Initial activity | 1 month | 2 months |
| Control = CNase | | | |
| 4° C. | 50 U/ml | 44 U/ml | 37 U/ml |
| 16° C. | 50 U/ml | 28 U/ml | 26 U/ml |
| 32° C. | 50 U/ml | 13 U/ml | 11 U/ml |
| CNase + Sepiret | | | |
| 4° C. | 36 U/ml | 32 U/ml | 27 U/ml |
| 16° C. | 36 U/ml | 31 U/ml | 27 U/ml |
| 32° C. | 36 U/ml | 21 U/ml | 13 U/ml |

It can be seen that the degree of recovery of the chitinolytic activity after drying and taking up into the buffer was equal to 36/50=72%. It can also be seen that while the loss of recoverable activity was identical at 4° C. (of the order of 25% after 2 months storage) between the control and enrobed batches, it remained much higher without Sepiret (48% loss at 16° C. instead of 25%, and 78% loss at 32° C. instead of 64%). It can be concluded that the enrobing agents help to stabilise the chitinases used.

References

K. Broglie, I. Chet, M. Holliday, R. Cressman, P. Biddle, C. Knowlton, C. J. Mauvais, R. Broglie (1991). Transgenic plants with enhanced resistance to the fungal pathogen Rhizoctonia solani. Science, 254, 1194-1197.

B. J. C. Comelissen and L. Melchers (1993). Strategies for control of fungal diseases with transgenic plants. Plant Physiol., 101, 709-712.

B. Digat (1994). Les bactéries stimulatrices de la croissance des plantes: le cas des Pseudomonas. C.R.Acad.Agric.Fr., 80, n⁰2, 125-140; meeting, Mar. 2^{nd}, 1994.

K. Düring (1993). Can Lysozymes mediate antibacterial resistance in plants? Plant Mol. Biol., 23, 209-214.

M. O Gilbert, Y. Y. Zhang, Z. K. Punja (1996). Introduction and expression of chitinase encoding genes in carrot following Agrobacterium-mediated transformation. In Vitro Cell. Dev. Biol.-Plant, 32, 171-178.

G. E. Harman, R. M. Broadway, A. Tronsmo, M. Lorito (1992). Purified Chitinases and Use Thereof. U.S. Pat. No. 5,173,419.

F. He et al., Canadian patent CN 1 044 748. Preparation and usage of seed-treating agent containing chitin (1990).

M. Hirano et al., Japanese patent JP 03 198 702 (91 198 702). Method for promoting seed germination (1991).

S. Hirano, T. Yamamoto, M. Hayashi, T. Nishida, H. Inui (1990). Chitinase activity in seeds coated with chitosan derivatives. Agric. Biol. Chem., 54, 10, 2719-2720.

H. Inui, Y. Yamaguchi, Y. Ishigami, S. Kawaguchi, T. Yamada, H. Ihara, S. Hirano (1996). Three extracellular chitinases in suspension-culture rice cells elicited by N-acetyl chitooligosaccharides. Biosci. Biotech. Biochem., 60, 12, 1956-1961.

G. Jach, S. Logemann, G. Wolf, A. Oppenheim, I. Chet, J. Schell, J.Logemann (1992). Expression of a bacterial chitinase leads to improved resistance of transgenic tobacco plants against fungal infection. Biopractice, 1, 1-10.

Y. Kajimura et al., Japanese patent JP 03 34 905 (91 34 905). Compositions containing chitinase-producing bacteria and colloidal chitin for growth acceleration and disease prevention in plants (1991).

J. M. Neuhaus, P. AhI-Goy, U. Hinz, S. Flores, F. Meins Jr (1991). High-level expression of a tobacco chitinase gene in Nicotiana sylvestris. Susceptibility of transgenic plants to Cercospora nicotianae infection. Plant Mo. Biol., 16, 141-151.

J. Rajnchapel-Messai (1988). La stimulation des défenses des plantes. Biofutur, April 1988, 67, 21-29.

M. Schirmböck, M. Lorito, Y.-L. Wang, C. K. Hayes, I. Arisan-Atac, F. Scala, G. E. Harman, C. P. Kubicek (1994). Parallel formation and synergism of hydrolytic enzymes and peptaibol antibiotics, molecular mechanisms involved in the antagonistic action of Trichoderma harzanium against phytopathogenic fungi. Appl. Environ. Microbiol., 60, 4364-4370.

A. Stintzi, T. Heitz, V. Prasad, S. Wiedemann-Merdinoglu, S. Kauffinann, P. Geoffroy, M. Legrand, B. Fritig (1993). Plant “pathogenesis-related” proteins and their role in defense against pathogens. Biochimie, 75, 687-706.

P. Teichgräber, L. Popper, D. Knorr (1991). Chitosan as an elicitor for the production of chitinase, an antifungal enzyme from soybean seeds. Agro Industry Hi-Tech, June 1991, 11-14.

J. Trudel, C. Potvin, A. Asselin (1995). Secreted hen lysozyme in transgenic tobacco : recovery of bound enzyme and in vitro growth inhibition of plant pathogens. Plant Sci., 106 (1) 55-62.

What is claimed is:

1. A fungicidal or fungistatic composition comprising at least one glycolytic enzyme and its substrate or oligomers of its substrate, the fungicidal or fungistatic composition being present in an amount effective for providing a germination inhibiting effect of less than about 10% when the composition is applied to a seed.

2. A fungicidal or fungistatic composition according to claim 1 wherein the glycolytic enzyme is a glycosidase selected from the group consisting of chitinases, laminarinases, and mixtures thereof.

3. A fungicidal or fungistatic composition according to claim 1 further comprising lysozyme.

4. A fungicidal or fungistatic composition according to claim 1 wherein the composition includes chitinase, laminarinase, lysozyme, and chitin or oligomers of chitin obtained by controlled hyrdolysis of chitin.

5. A fungicidal or fungistatic composition according to claim 4 wherein the chitinase is obtained from Serratia marcescens.

6. A fungicidal or fungistatic composition according to claim 4 wherein the laminarinase is obtained from Bacillus circulans.

7. A fungicidal or fungistatic composition according to claim 1 wherein the substrate is chitin or oligomers of chitin obtained by controlled hydrolysis of chitin.

8. A fungicidal or fungistatic composition according to claim 7 wherein oligomers originating from chitin have a formula [N-acetylglucosamine]ₙ, wherein n is 1 to 8.

9. A fungicidal or fungistatic composition according to claim 1 having a weight ratio of enzyme to substrate of about 1:10 to about 10:1.

10. A fungicidal or fungistatic composition according to claim 1 wherein the composition is incorporated into a film-forming preparation.

11. A fungicidal or fungistatic composition according to claim 10 wherein the film-forming preparation is a pelliculisation, enrobing or encapsulation agent.

12. A process for preparing a biofungicidal composition comprising:
producing chitinase from Serratia marcescens;
producing laminarinase from Bacillus circulans;
preparing colloidal chitin; and
incorporating the chitinase, laminarinase and colloidal chitin into a film-forming preparation.

13. A process for preparing a biofungicidal composition according to claim 12 wherein lysozyme is incorporated into the film-forming preparation.

14. A process for preparing a biofungicidal composition according to claim 12 wherein the chitin has been partially hydrolyzed.

15. A process for preparing a biofungicidal composition according to claim 12 wherein the film-forming preparation is a pelliculisation, enrobing or encapsulation agent.

16. A method for protecting seeds, bulbs or roots against fungicidal infections comprising treating the seeds, bulbs or roots with a fungicidal or fungistatic composition comprising at least one glycolytic enzyme and its substrate or oligomers of its substrate.

17. A method for protecting seeds from fungicidal infections comprising treating the seeds with a fungicidal or fungistatic composition comprising at least one glycolytic enzyme and its substrate or oligomers of its substrate, the fungicidal or fungistatic composition being present in an amount effective for providing a germination inhibiting effect of less than about 10% when the composition is applied to a seed.

18. A method for treatment of food packaging comprising treating the food packaging with a fungicidal or fungistatic composition comprising at least one glycolytic enzyme and its substrate or oligomers of its substrate.

19. A strain of Serratia marcescens having accession number LMG P-18541.

20. A film-forming preparation comprising:
a fungicidal or fungistatic composition, the fungicidal or fungistatic composition comprising at least one glycolytic enzyme and its substrate or oligomers of its substrate; and
a pelliculisation, enrobing or encapsulating agent.
